(19) United States
(12) Patent Application Publication
(10) Pub. No.: US 2025/0185927 A1
(43) Pub. Date: Jun. 12, 2025

(54) CUFF STRUCTURE AND BLOOD PRESSURE MEASUREMENT DEVICE

(71) Applicant: OMRON HEALTHCARE Co., Ltd., Kyoto (JP)

(72) Inventors: Yoshihide TOKKO, Kyoto (JP); Masaki HARADA, Kyoto (JP); Yoshihiko SANO, Kyoto (JP); Yuki TAKANO, Kyoto (JP)

(21) Appl. No.: 19/058,769

(22) Filed: Feb. 20, 2025

Related U.S. Application Data
(63) Continuation of application No. PCT/JP2023/031201, filed on Aug. 29, 2023.

(30) Foreign Application Priority Data
Dec. 22, 2022 (JP) 2022-205637

(51) Int. Cl. A61B 5/021

(52) U.S. Cl. CPC A61B 5/02141

(57) ABSTRACT

A cuff structure for use in a blood pressure measurement device includes one or more air bags that are inflated by a fluid and one or more members that are included in the air bags or are laminated on the air bags, in which the air bags and at least two of the one or more members are provided with bent portions, one of which is formed by an opening or a division and the other of which is formed by an opening, a division, a notch, a recessed portion, or a groove.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/031201, filed Aug. 29, 2023, which application claims priority to Japanese Patent Application No. 2022-205637, filed Dec. 22, 2022, which applications are incorporated herein by reference in their entireties.

### TECHNICAL FIELD

The present invention relates to a cuff structure and a blood pressure measurement device.

### BACKGROUND ART

In recent years, blood pressure measurement devices used for measuring a blood pressure are used as means to check health status at home, as well as in medical facilities. A blood pressure measurement device detects vibration of the artery wall to measure blood pressure by, for example, inflating and contracting a cuff wound around the upper arm or the wrist of a living body and detecting the pressure in the cuff by using a pressure sensor.

As a cuff used in such a blood pressure measurement device, a cuff having a laminated structure is known as described in JP 2001-224558 A. In the cuff having the laminated structure, a coupling portion is provided and a side wall portion is made hard to prevent the cuff from being laterally bulged, and the cuff is achieved in which the width of the cuff does not change even when the cuff is inflated and compression can be efficiently performed. On the other hand, the cuff having the laminated structure has a problem in that the provision of the coupling portion inhibits a gusset portion from bulging outward, resulting in a trade-off between a compression efficiency and an ease with which the cuff is laterally bulged.

In addition, it is generally difficult to adjust a material intensity, i.e., to change the thickness and hardness of the sheet member forming the cuff, and this leads to an intensity adjustment of the entire cuff, so that it is impossible to increase the intensity of the cuff only at a portion where the lateral bulge is likely to be generated. In addition, the cuff having the laminated structure is formed by laminating a large number of sheet members, and thus there is a problem that the cuff is thick and wrinkles are likely to be generated. There is a problem in that when the wrinkles are generated in the cuff, the space inside the cuff is divided and an accuracy of blood pressure measurement decreases.

Thus, as a conventional wrinkle control method of the cuff, as disclosed in JP 6751462 B, there is known a technology for controlling wrinkles by providing a notch in a partition sheet. This technology aims at reducing stress concentration by forming a largely bent wrinkle as a gentle wrinkle.

### CITATION LIST

### Patent Literature

Patent Literature 1: JP 2001-224558 A
Patent Literature 2: JP 6751462 B

### SUMMARY OF INVENTION

### Technical Problem

In the wrinkle control method of the cuff described above, when the cuff is wound around a measurement site having a small curvature, wrinkles are also generated at positions other than a position where the notch is provided, and a sufficient effect cannot be exhibited. Further, when the cuff itself has a thickness as in the case of the above-described laminated structure, a sufficient effect cannot be exhibited due to the thickness.

Thus, an object of the present invention is to provide a cuff structure that can control the generated wrinkles and a blood pressure measurement device.

### Solution to Problem

According to one aspect, there is provided a cuff structure for use in a blood pressure measurement device, the cuff structure including one or more air bags that are inflated by a fluid and one or more members that are included in the air bags or are laminated on the air bags, wherein the air bag and at least two of the one or more members are provided with bent portions, one of which is formed by an opening or a division and the other of which is formed by an opening, a division, a notch, a recessed portion, or a groove.

According to this aspect, the intensities of the two or more members are weakened by the bent portion including the opening, and thus the cuff structure can control wrinkles generated in the curved air bag when the cuff structure is attached to the living body. Thus, the cuff structure can generate desired wrinkles, unintended wrinkles such as excessively deep wrinkles can be prevented from being generated. Thus, the cuff structure can stably perform blood pressure measurement using the blood pressure measurement device.

There is provided the cuff structure according to the above-described aspect, the cuff structure including, a curler that is the member and is curved following a shape of a site of a living body and attached thereto in a circumferential direction, a pressing cuff that is fixed to an inner circumferential surface of the curler and includes the air bag, a back plate that is the member and is fixed to an inner circumferential surface of the pressing cuff, and a sensing cuff that is fixed to an inner circumferential surface of the back plate and includes the air bag.

According to this aspect, it is possible to control wrinkles of the pressing cuff and the sensing cuff that compress the living body and are greatly affected by stress concentration and a decrease in compression efficiency, and blood pressure measurement can be stably performed.

There is provided the cuff structure according to the above-described aspect, in which the air bag includes an intermediate sheet member, and the bent portion formed in the air bag is one or more openings formed in the intermediate sheet member and/or a recessed portion formed on a front surface of the air bag.

According to this aspect, the cuff structure can reduce the intensity of the air bag in which the wrinkles are generated, and thus the wrinkles generated in the air bag can be easily controlled. As a result, the cuff structure can reduce the influence on controlling the wrinkles due to the hardness of the air bag.

There is provided the cuff structure according to the above-described aspect, in which the bent portion formed in the back plate is an opening, a division, a notch, or a groove formed in the back plate.

According to this aspect, the cuff structure can reduce the intensity of the back plate to which the sensing cuff is fixed, and thus the wrinkles generated in the sensing cuff can be easily controlled.

There is provided the cuff structure according to the above-described aspect, in which the curler, the pressing cuff, the back plate, and the sensing cuff are formed to be long in one direction, and a plurality of the bent portions provided in at least two of the curler, the pressing cuff, the back plate, and the sensing cuff are provided side by side in the one direction.

According to this aspect, the cuff structure is provided with the plurality of bent portions side by side in one direction, and thus the cuff structure can disperse the wrinkles generated in the air bag. The cuff structure is provided with the wrinkles in a dispersed manner, and thus generation of deep wrinkles can be suppressed.

There is provided the cuff structure according to the above-described aspect, in which the bent portions provided in at least two of the curler, the pressing cuff, the back plate, and the sensing cuff are disposed to at least partially overlap the bent portions adjacent to each other in a lamination direction and/or disposed to be shifted from the bent portions adjacent to each other in the lamination direction in the one direction.

According to this aspect, the bent portions provided in the two or more members are at least partially overlapped each other, and thus the wrinkle can be generated at a target predetermined position. In addition, the bent portions provided in the two or more members are shifted from each other, and thus the wrinkles can be finely dispersed. As described above, the cuff structure can set any positions and the number of the wrinkles by disposing the bent portions.

According to one aspect, there is provided a blood pressure measurement device including the cuff structure according to the one aspect, a device body fixed to the cuff structure, and a band provided in the device body and fixing the cuff structure.

According to this aspect, the blood pressure measurement device includes the cuff structure, and thus the variation in the generation of the wrinkles due to a state at the time of attachment or the shape of the wrist can be suppressed and the variation in the measurement accuracy of the blood pressure caused by the wrinkles can be suppressed.

### Advantageous Effects of Invention

The present invention can provide a cuff structure that can control the generated wrinkles and a blood pressure measurement device.

### BRIEF DESCRIPTION OF DRAWINGS

FIG. 1 is a perspective view illustrating a configuration of a blood pressure measurement device according to an embodiment of the present invention.

FIG. 2 is a side view illustrating the configuration of the blood pressure measurement device.

FIG. 3 is a side view illustrating the configuration of the blood pressure measurement device in a state of being attached to the wrist.

FIG. 4 is a block diagram illustrating the configuration of the blood pressure measurement device.

FIG. 5 is an explanatory view schematically illustrating a configuration of a cuff structure used in the blood pressure measurement device in a developed manner from a side surface side.

FIG. 6 is a cross-sectional view schematically illustrating a configuration of a cuff used in the cuff structure.

FIG. 7 is a cross-sectional view schematically illustrating another example of the cuff.

FIG. 8 is a cross-sectional view schematically illustrating another example of the cuff.

FIG. 9 is a plan view schematically illustrating a configuration of a bent portion used in the cuff structure.

FIG. 10 is a plan view schematically illustrating a configuration of another example of the bent portion.

FIG. 11 is a plan view schematically illustrating a configuration of another example of the bent portion.

FIG. 12 is a plan view schematically illustrating a configuration of another example of the bent portion.

FIG. 13 is a plan view schematically illustrating a configuration of another example of the bent portion.

FIG. 14 is a plan view schematically illustrating a configuration of another example of the bent portion.

FIG. 15 is a plan view schematically illustrating a configuration of another example of the bent portion.

FIG. 16 is a plan view schematically illustrating a configuration of another example of the bent portion.

FIG. 17 is a cross-sectional view schematically illustrating a configuration of another example of the bent portion.

FIG. 18 is a plan view schematically illustrating a configuration of another example of the bent portion.

FIG. 19 is a plan view schematically illustrating a configuration of another example of the bent portion.

FIG. 20 is a plan view schematically illustrating a configuration of another example of the bent portion.

FIG. 21 is a plan view schematically illustrating a configuration of another example of the bent portion.

FIG. 22 is a plan view schematically illustrating a configuration of the bent portion used in the cuff structure in a developed manner.

FIG. 23 is a plan view schematically illustrating a configuration of the bent portion used in the cuff structure in a developed manner.

FIG. 24 is an explanatory view illustrating an evaluation result of the cuff structure in comparison with comparative examples.

### DESCRIPTION OF EMBODIMENTS

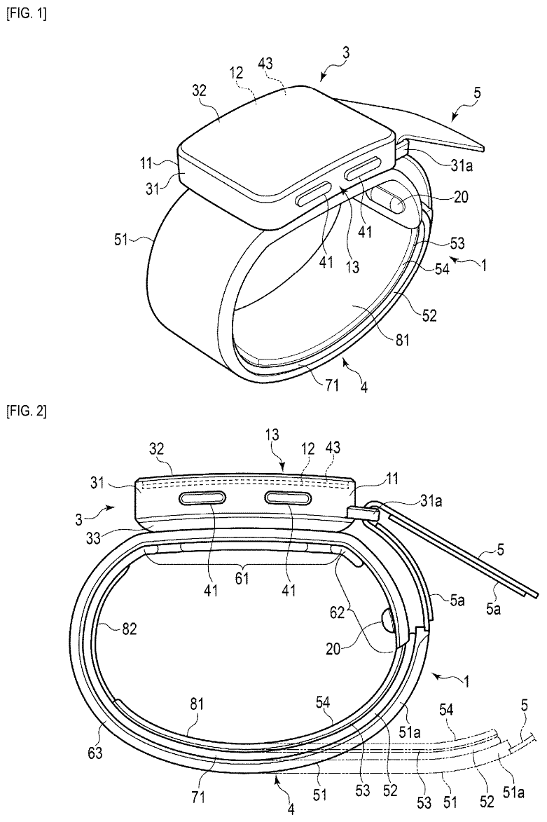

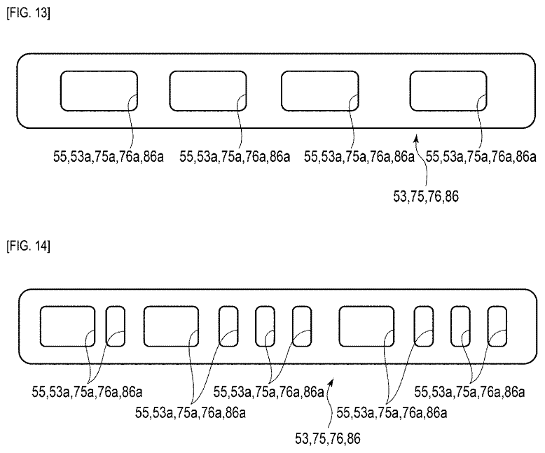

Hereinafter, an example of a blood pressure measurement device 1 according to an embodiment of the present invention will be described with reference to FIG. 1 to FIG. 24.

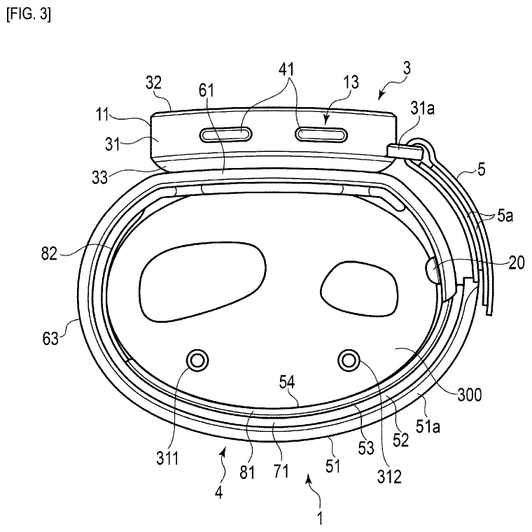

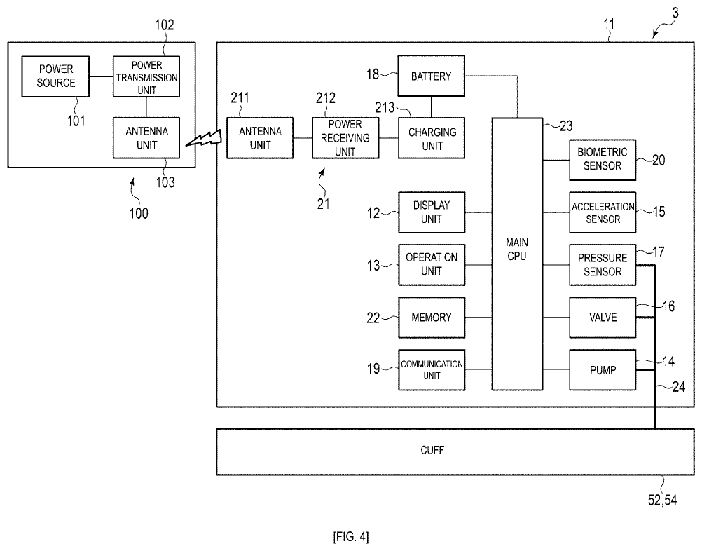

FIG. 1 is a perspective view illustrating a configuration of the blood pressure measurement device 1 according to the embodiment of the present invention, and FIG. 2 is a side view illustrating the configuration of the blood pressure measurement device 1. FIG. 3 is a side view illustrating the configuration of the blood pressure measurement device 1 in a state of being attached to a wrist 300. FIG. 4 is a block diagram illustrating a configuration of a device body 3 of the blood pressure measurement device 1.

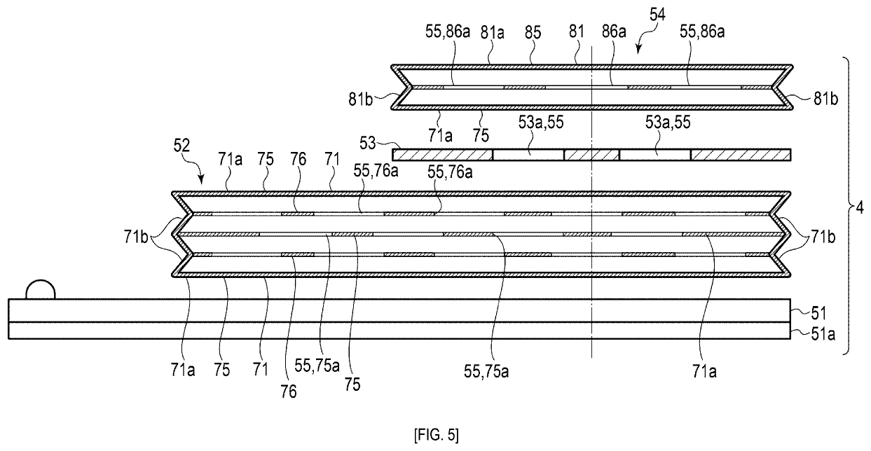

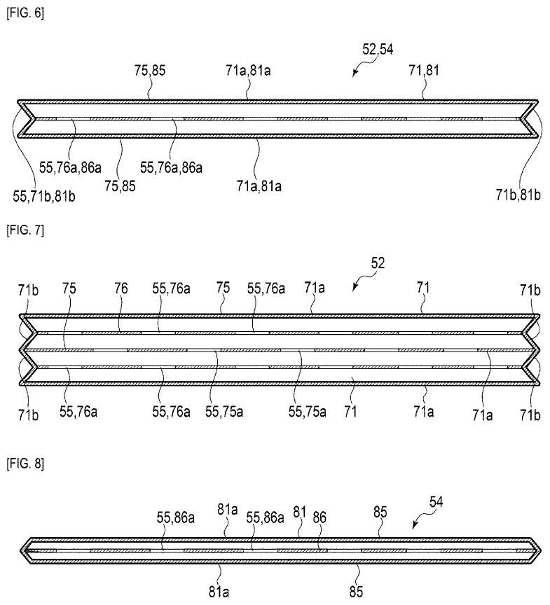

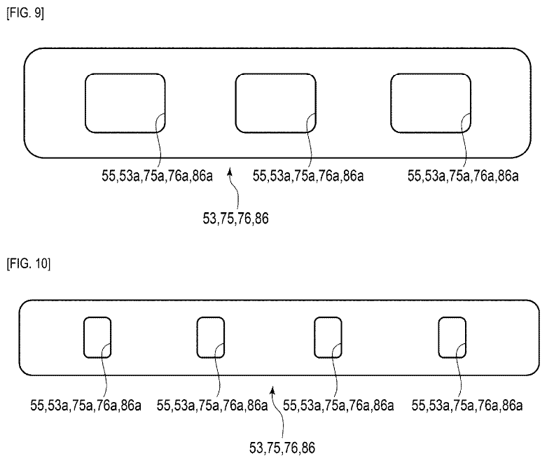

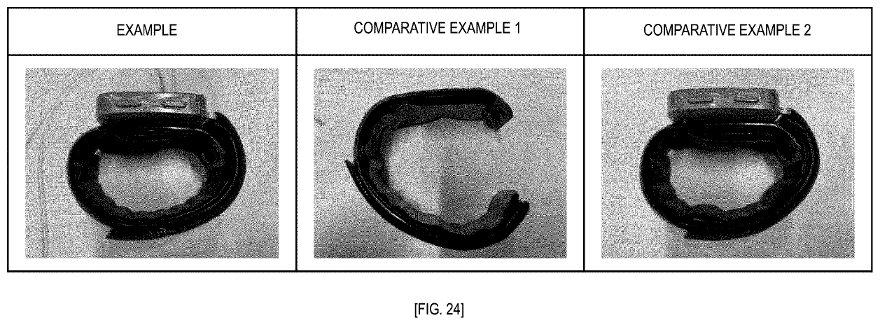

FIG. 5 is an explanatory view schematically illustrating a configuration of a cuff structure 4 used in the blood pressure measurement device 1 in a developed manner from a side surface side. FIG. 6 is a cross-sectional view schematically illustrating configurations of a pressing cuff 52 and a sensing cuff 54 as cuffs used in the cuff structure 4. FIG. 7 is a cross-sectional view schematically illustrating a configuration of another example of the pressing cuff 52. FIG. 8 is a cross-sectional view schematically illustrating a configuration of another example of the sensing cuff 54. FIGS. 9 to 21 are views schematically illustrating examples of a bent portion 55 provided in each member of the cuff structure 4. FIG. 24 is an explanatory view illustrating an evaluation result of the cuff structure 4 in comparison with comparative examples.

The blood pressure measurement device 1 is an electronic blood pressure measurement device attached to a living body. In an example of the present embodiment, the blood pressure measurement device 1 is a wearable device attached to the wrist 300 as illustrated in FIG. 3. The blood pressure measurement device 1 is, for example, an electronic blood pressure measurement device having an aspect of measuring a blood pressure from arteries 311 and 312 of the wrist 300.

As illustrated in FIGS. 1 to 3, the blood pressure measurement device 1 includes the device body 3, the cuff structure 4, and a band 5.

As illustrated in FIGS. 1 to 4, the device body 3 includes, for example, a housing 11, a display unit 12, an operation unit 13, a pump 14, an acceleration sensor 15, a valve 16, a pressure sensor 17, a battery 18, a communication unit 19, a charging circuit unit 21, a memory 22, and a processor 23.

The housing 11 is a case that accommodates components of the device body 3. The housing 11 accommodates, for example, the display unit 12, the operation unit 13, the pump 14, the acceleration sensor 15, the valve 16, the pressure sensor 17, the battery 18, the communication unit 19, a biometric sensor 20, the charging circuit unit 21, the memory 22, and the processor 23. The housing 11 accommodates, for example, a fluid circuit 24. Here, the fluid circuit 24 may include, for example, a pipe or a flow path plate that forms a flow path of a fluid supplied from the pump 14 to the cuff structure 4, a component such as a fluid resistor that controls a supply amount or a pressure of the fluid supplied to the cuff structure 4, and a check valve that controls a flow direction of the fluid.

The housing 11 includes, for example, an outer case 31, a windshield 32 that covers an upper opening of the outer case 31, and a back lid 33 that covers a lower portion of the outer case 31.

The outer case 31 is formed in, for example, a cylindrical shape, a rectangular cylindrical shape, or a polygonal cylindrical shape. In the present embodiment, the outer case 31 is formed in the rectangular cylindrical shape. The outer case 31 includes a loop portion 31a provided on one surface of an outer circumferential surface. The loop portion 31a is a rectangular annular member including an opening that is long in one direction and through which the band 5 can be inserted in order to allow the band 5 to pass the loop portion 31a and to fold back the band 5. The loop portion 31a is integrally formed with the outer case 31. The windshield 32 is a glass plate having the same shape as that of an outer circumferential edge of the outer case 31, that is, a rectangular shape in the present embodiment. The windshield 32 is not limited to the glass plate as long as the windshield 32 is made of a transparent or translucent material. The back lid 33 closes the bottom of the outer case 31. The housing 11 may have a configuration in which the back lid 33 is not included, and the bottom of the outer case 31 may be closed by a curler 51 (described later) of the cuff structure 4, the curler 51 being fixed to the housing 11.

The display unit 12 is disposed directly below the windshield 32. The display unit 12 is electrically connected to the processor 23. The display unit 12 is, for example, a liquid crystal display or an organic electroluminescence display. The display unit 12 displays various types of information including the date and time, measurement results of blood pressure values such as the systolic blood pressure and diastolic blood pressure, heart rate, a charging status and a remaining amount of the battery 18, and the like. For example, the display unit 12 is formed in the same shape as the windshield 32 in a plan view.

The operation unit 13 is configured to be capable of inputting a command from a user. The operation unit 13 includes, for example, a plurality of buttons 41 provided on the housing 11, a sensor that detects operation of each of the buttons 41, and a touch panel 43 provided on the display unit 12 or the windshield 32. When the operation unit 13 is operated by the user, the operation unit 13 converts a command into an electrical signal. The sensor and the touch panel 43 are electrically connected to the processor 23 to output electrical signals to the processor 23.

The pump 14 is, for example, a piezoelectric pump. The pump 14 compresses air as a fluid, for example, and supplies the compressed air to the pressing cuff 52 and the sensing cuff 54, which are described later, of the cuff structure 4 via the fluid circuit. The pump 14 is electrically connected to the processor 23.

The acceleration sensor 15 is, for example, a 3-axis acceleration sensor. The acceleration sensor 15, for example, measures acceleration and outputs an analog signal. The acceleration sensor 15 is, for example, connected to the processor 23 via an A/D conversion circuit.

The valve 16 is, for example, an on-off valve. The valve 16 opens and closes a fluid circuit connecting the pump 14 to the cuff structure 4 and/or a fluid circuit connecting the cuff structure 4 to the atmosphere. The valve 16 is electrically connected to the processor 23. For example, the valve 16 is opened and closed by control of the processor 23.

As a specific example, the valve 16 is a safety valve that releases air supplied to the pressing cuff 52 and the sensing cuff 54 (which are described later) of the cuff structure 4 to the atmosphere. The valve 16 is switched to a closed state by being controlled by the processor 23 when air is supplied to the pressing cuff 52 and the sensing cuff 54 during a blood pressure measurement. In addition, when the air in the pressing cuff 52 and the sensing cuff 54 is exhausted, the valve 16 is controlled by the processor 23 so as to be switched from the closed state to the open state. Further, the valve 16 may be formed such that the opening degree is adjustable. The valve 16 may be provided on the fluid circuit 24, or may be integrally provided inside a housing of the pump 14.

The pressure sensor 17 is provided, for example, in the fluid circuit 24. The pressure sensor 17 detects the pressures of the pressing cuff 52 and/or the sensing cuff 54. For example, the pressure sensor 17 detects the pressure of the sensing cuff 54. The pressure sensor 17 is electrically connected to the processor 23 via the A/D conversion circuit, converts detected pressure into an electrical signal, and outputs the electrical signal to the processor 23.

The battery 18 is, for example, a secondary battery such as a chargeable and dischargeable lithium ion battery. The battery 18 is electrically connected to the processor 23. The battery 18 supplies power to the processor 23. The battery 18 supplies power for driving to the respective configurations of the processor 23 and the display unit 12, the operation unit 13, the pump 14, the acceleration sensor 15, the valve 16, the pressure sensor 17, and the communication unit 19 via the processor 23.

The communication unit 19 can transmit and receive information to and from an external device wirelessly and/or by wire. The communication unit 19 is, for example, a wireless communication module conforming to a wireless communication standard. The communication unit 19 transmits information, such as information controlled by the processor 23 and measured blood pressure values and pulse, to an external device, or receives, for example, a program or recording medium for software update from an external device and transmits this to the control unit. In the present embodiment, the external device is, for example, an external terminal, such as a smartphone, a tablet terminal, a personal computer, and a smart watch.

In the present embodiment, the communication unit 19 and the external terminal may be directly connected, or may be connected over a network. The communication unit 19 and the external terminal may be connected via a mobile communication network, such as 4G and 5G, and a wireless communication line, such as Wimax and Wi-Fi (registered trademark). Further, the communication unit 19 and the external device may be connected by wireless communication means, such as Bluetooth Low Energy (BLE (registered trademark)), Near Field Communication (NFC), and infrared communication. Furthermore, in addition to the wireless communication module, the communication unit 19 may include a universal connector such as a micro Universal Serial Bus (USB) or a dedicated connector for the blood pressure measurement device 1, and may be directly connected to the external terminal by various cables such as a USB cable or connected to the external terminal via a wired communication line such as a Local Area Network (LAN) connection. Thus, the communication unit 19 may include a plurality of communication means, such as a wireless antenna and a micro-USB connector. The connector for wired communication may be a dedicated connector for the blood pressure measurement device 1.

The biometric sensor 20 is formed so as to be capable of detecting information on a living body by coming into contact with or facing the wrist 300. The biometric sensor 20 converts the detected information on a living body into an electrical signal and outputs the electrical signal to the processor 23. The biometric sensor 20 may be, for example, a sensor that measures a physical quantity such as a heart rate or a body temperature, or may be a sensor that measures a chemical value such as a blood glucose level or a blood oxygen concentration. For example, the biometric sensor 20 is provided on the back lid 33 of the housing 11 and/or the curler 51 (described later) of the cuff structure 4. In the present embodiment, an example is illustrated in which the biometric sensor 20 is provided on the curler 51. The blood pressure measurement device 1 need not include the biometric sensor 20.

The charging circuit unit 21 includes, for example, an antenna unit 211, a power receiving unit 212, and a charging unit 213. The charging circuit unit 21 charges the battery 18 by wireless power feeding. For example, the charging circuit unit 21 receives transmission power transmitted from the antenna unit 103 of a power transmission device 100 provided outside and charges the battery 18.

The antenna unit 211 receives the transmission power from the antenna unit of the power transmission device. The antenna unit 211 is, for example, a receiver coil as a power receiving resonance circuit. The antenna unit 211 supplies the received power to the power receiving unit 212. A power receiving surface of the antenna unit 211 is formed in a planar shape. The antenna unit 211 is disposed, for example, in the housing 11. As a specific example, the antenna unit 211 is provided in the housing 11 and adjacent to the display unit 12 on a side opposite to the windshield 32 of the display unit 12. The antenna unit 211 includes, for example, a resonance capacitor and constitutes the power receiving resonance circuit.

The power receiving unit 212 rectifies the power received by the antenna unit 211 and supplies the rectified power to the charging unit 213. As a specific example, the power receiving unit 212 rectifies the received power supplied from the antenna unit 211 and converts rectified received power from AC to DC. For example, the power receiving unit 212 includes a rectifying circuit and a control circuit, controls the operation of the rectifying circuit by the control circuit, and outputs rectified DC power to the charging unit 213.

The charging unit 213 supplies the power supplied from the power receiving unit 212 to the battery 18 as power for charging. For example, the charging unit 213 converts the power supplied from the power receiving unit 212 into a predetermined current value and a predetermined voltage value and supplies the converted power to the battery 18. Further, for example, the charging unit 213 may include a circuit that outputs the charge state of the battery 18 to the power receiving unit 212 and/or the processor 23.

The memory 22 includes, for example, a Random Access Memory (RAM) and a Read Only Memory (ROM). The memory 22 stores various data. For example, the memory 22 stores in advance, for example, programs or recording mediums and various program or recording medium data such as applications for controlling the entire blood pressure measurement device 1 and the pump 14, settings data for setting various functions of the blood pressure measurement device 1, and calculation data for calculating a blood pressure value and a pulse from the pressure measured by the pressure sensor 17 to be changeable.

The processor 23 controls the operation of the entire blood pressure measurement device 1 and the operations of the pump 14 and the valve 16 based on the programs or recording mediums stored in the memory 22 to perform a predetermined operation (function). The processor 23 executes the predetermined operation, analysis, processing, or the like according to the read program or recording medium. The processor 23 is an arithmetic device such as a CPU. The processor 23 may include, for example, a sub CPU in addition to a main CPU. In addition, the processor 23 displays statuses or results of the executed various operations, calculations, analyses, processes, and the like on the display unit 12 by the program (or recording medium) or the application.

The cuff structure 4 includes, for example, the curler 51, the pressing cuff 52, the back plate 53, the sensing cuff 54 and the bent portion 55. The cuff structure 4 is formed by layering the curler 51, the pressing cuff 52, the back plate 53, and the sensing cuff 54. In the cuff structure 4, the bent portion 55 is formed in at least two members among the curler 51, the pressing cuff 52, the back plate 53, and the sensing cuff 54. which are the plurality of members constituting the cuff structure 4. In addition, for example, the cuff structure 4 may be configured such that the bent portion 55 is formed in a plurality of members constituting the pressing cuff 52 and/or the sensing cuff 54, for example, sheet members 75 and 85 and intermediate sheet members 76 and 86 (which are described later).

Hereinafter, specific examples of the cuff structure 4 will be described with reference to FIGS. 1 to 3 and 5 to 23. As illustrated in FIGS. 1 to 3 and 5, the cuff structure 4 includes the curler 51, the pressing cuff 52, the back plate 53, the sensing cuff 54, and a plurality of the bent portions 55 formed in each of the pressing cuff 52, the back plate 53, and the sensing cuff 54.

For example, a first end side of the curler 51 is fixed to the wrist side of the housing 11. The curler 51 is formed in a band-like shape that curves in a shape following along the circumferential direction of the wrist 300. The curler 51 is constituted by a resin material. In addition, the curler 51 includes a decorative sheet 51a such as an outer cloth provided on the outer circumferential surface side by welding, bonding, integral molding, or the like for improving design. The curler 51 has hardness appropriate to provide flexibility and shape retainability. Here, “flexibility” refers to deformation of the shape of the curler 51 in a radial direction at the time of application of an external force of the band 5 to the curler 51. “Shape retainability” refers to the ability of the curler 51 to maintain a pre-imparted shape when no external force is applied to the curler 5. That is, the curler 51 is made of a resin material having a hardness that does not undergo compressive deformation or substantially does not undergo compressive deformation but allows elastic deformation such as bending deformation in which the shape, in particular, a curvature of a curved portion changes. Thus, the curler 51 is formed to be elastically deformable so that an inner space in which the wrist 300 is disposed becomes larger or smaller following the shape of the wrist to which the curler 51 is attached by the application of the external force.

In addition, the curler 51 is formed such that a first side from a portion fixed to the housing 11 is longer than a second side. For example, both ends of the curler 51 are formed to have a length and a shape so as to be located at a first side portion of the wrist 300 between a palm side of a hand and a back side of the hand of the wrist 300 even when the curler 51 is attached to either the wrist 300 having the longest circumference or the wrist 300 having the shortest circumference among users to whom the curler 51 is expected to be attached. In addition, the curler 51 may have a shape in which the entirety of the curler 51 curved in the longitudinal direction so as to follow the shape of the wrist 300 or part of the curler 51 is partially formed in a flat plate shape.

As a specific example, the curler 51 is, for example, fixed to the outer case 31 or the back lid 33 of the housing 11. The curler 51 includes a fixed portion 61 in which a portion fixed to the back lid 33 is formed in a flat plate shape, a first curved portion 62 provided at a first end of the fixed portion 61 and curved with a predetermined radius of curvature, and a second curved portion 63 provided at a second end of the fixed portion 61 and curved with a predetermined radius of curvature.

The first curved portion 62 is formed to have a length from the fixed portion 61 to the first side portion of the wrist 300. In addition, for example, the biometric sensor 20 is provided on the inner surface of the end portion side of the first curved portion 62. The second curved portion 63 is formed to have a length from the fixed portion 61 to the first side portion of the wrist 300 on which the end portion of the first curved portion 62 is disposed, beyond a second side portion of the wrist 300 and the palm side of the hand of the wrist 300.

For example, one of the end portion of the first curved portion 62 and the end portion of the second curved portion 63 is located further outward in the radial direction than the other. As a specific example, as illustrated in FIGS. 2 and 3, the end portion of the second curved portion 63 is located further outward in the radial direction than the end portion of the first curved portion 62. For example, as indicated by a two dot chain line in FIG. 2, in a state before the external force is applied to the curler 51, the end portion of the second curved portion 63 may be located further outward in the radial direction than the end portion of the first curved portion 62 and separated in the circumferential direction, and the end portions of the first curved portion 62 and the second curved portion 63 may be separated from each other.

For example, the lengths of the first curved portion 62 and the second curved portion 63 are lengths by which when the blood pressure measurement device 1 is attached to the wrist 300 having the longest circumference among the wrists 300 to which the blood pressure measurement device 1 is expected to be attached, the second curved portion 63 faces a site of the wrist 300 where the two arteries 311 and 312 are present, the second curved portion 63 is disposed on a part of the side portion of the wrist 300, and the end portion of the first curved portion 62 and the end portion of the second curved portion 63 are separated from each other. The two arteries 311 and 312 as used herein are the radial artery 311 and the ulnar artery 312.

Further, for example, the lengths of the first curved portion 62 and the second curved portion 63 are lengths by which when the blood pressure measurement device 1 is attached to the wrist 300 having the shortest circumference among the wrists 300 to which the blood pressure measurement device 1 is expected to be attached, the second curved portion 63 faces a site of the wrist 300 where the two arteries 311 and 312 are present, and the first curved portion 62 and the second curved portion 63 overlap each other at the side portion of the wrist 300.

The curler 51 has the largest curvature at a boundary (ridge portion) between the fixed portion 61 and the first curved portion 62 and at a boundary (ridge portion) between the fixed portion 61 and the second curved portion 63.

The pressing cuff 52 is fixed to the inner circumferential surface of the curler 51 by a double-sided tape, an adhesive, thermal welding, or the like. The pressing cuff 52 is provided at least in a region of the second curved portion 63 where the artery of the wrist 300 is present. As a specific example, the pressing cuff 52 is provided in a region of the curler 51 in the longitudinal direction from the fixed portion 61 side of the first curved portion 62 including the ridge portion between the fixed portion 61 and the first curved portion 62 to the end portion side of the second curved portion 63. The pressing cuff 52 extends along an inner surface of the curler 51 and has the largest curvature at the boundary between the fixed portion 61 and the first curved portion 62 and at the boundary between the fixed portion 61 and the second curved portion 63.

The pressing cuff 52 is fluidly connected to the pump 14 through the fluid circuit. A first main surface of the pressing cuff 52 is fixed to the inner surface of the curler 51. For example, the pressing cuff 52 is affixed to the inner surface of the curler 51 by a double-sided tape of an adhesive. The pressing cuff 52 is inflated to press the back side of the hand of the wrist 300 and press the back plate 53 and the sensing cuff 54 toward the wrist 300 side.

The pressing cuff 52 includes, for example, a single or a plurality of air bags 71, and a connecting portion such as a nipple provided in the air bag 71 and connected to the fluid circuit 24. Here, the air bag 71 is a bag-like structure, and in the present embodiment, the blood pressure measurement device 1 is configured to use air with the pump 14, and thus the present embodiment will be described using the air bag. However, in a case where a fluid other than air is used, the bag-like structure may be any fluid bag that is inflated by the fluid. The air bag 71 is formed in a rectangular bag shape that is long in one direction.

For example, the air bag 71 includes a plurality of the sheet members 75 and a single intermediate sheet member 76 in which an opening 76a is formed in at least a part thereof. By fixing the plurality of sheet members 75 and the single or a plurality of the intermediate sheet members 76 by thermal welding or the like, the air bag 71 is formed in a bag shape in which an inner space is partitioned by the intermediate sheet member 76 and is partitioned into two chambers that fluidly communicate with each other by the intermediate sheet member 76.

The air bag 71 has, for example, a cuff structure in which a side wall is recessed in a 2 shape. The air bag 71 includes a pair of main walls 71a each formed by a part of a respective one of the sheet members 75 in the thickness direction, and a pair of side walls 71b each formed by an end portion in the lateral direction of a respective one of the sheet members 75 in the lateral direction.

The sheet member 75 forms, for example, the pair of main walls 71a in the thickness direction of the air bag 71 and a part of the pair of side walls 71b of the air bag 71. A central side of the side wall 71b is recessed in the >shape toward inward of the air bag 71 in the thickness direction of the air bag 71 (in the radial direction of the curler 51).

As illustrated in FIG. 6, when the single air bag 71 is provided in the pressing cuff 52, the curler 51 and the back plate 53 are fixed to the main walls 71a. When the plurality of air bags 71 are provided in the pressing cuff 52 as illustrated in FIG. 7, the plurality of air bags 71 are layered and integrally formed, the curler 51 is fixed to one of the main walls 71a of the air bag 71 on a first side among the plurality of air bags 71 in the thickness direction, and the back plate 53 is fixed to a part of one of the main 71a walls of the air bag 71 on a second side in the thickness direction. In addition, as illustrated in FIG. 7, the main walls 71a of adjacent ones of the air bags 71 among the plurality of air bags 71 are integrally fixed to each other by welding or the like, or are shared, and a single or a plurality of openings 75a that cause the adjacent ones of the air bags 71 to fluidly communicate with each other are formed. In other words, as illustrated in FIG. 7, the single or the plurality of openings 75a that cause the adjacent ones of the air bags 71 to fluidly communicate with each other are formed in the sheet member 75 that forms the main walls 71a facing each other or the shared main walls 71a of the adjacent ones of the air bags 71.

The plurality of sheet members 75 are welded to form the air bag 71 including the pair of main walls 71a and the pair of side walls 71b. Here, one air bag 71 may be formed by two sheet members 75, or may be formed by three or more sheet members 75, for example, four sheet members 75 constituting the main walls 71a and the side walls 71b. That is, the number of the sheet members 75 for forming the air bag 71 is not limited.

In addition, in a case where the plurality of air bags 71 are integrally layered, the sheet members 75 forming the main walls 71a of the adjacent ones of the air bags 71 may be welded to each other, or the main walls 71a of the adjacent ones of the air bags 71 may be formed by one sheet member 75 and the adjacent ones of the air bags 71 may share one main wall 71a.

The intermediate sheet member 76 is provided at a portion located at the most inward portion of the side wall 71b. The intermediate sheet member 76 partitions the inside of the air bag 71 into two chambers (spaces) in the thickness direction of the air bag 71. Further, in the intermediate sheet member 76, a single or a plurality of the openings 76a that fluidly communicate the two chambers in the air bag 71 are formed.

For example, a thermoplastic elastomer is used as the material of the sheet member 75 and the intermediate sheet member 76. Examples of thermoplastic elastomer includes thermoplastic polyurethane-based resin (hereinafter referred to as TPU), polyvinyl chloride resin, ethylene-vinyl acetate resin, thermoplastic polystyrene-based resin, thermoplastic polyolefin resin, thermoplastic polyester-based resin, and thermoplastic polyamide resin. As the thermoplastic elastomer used for the sheet member 75 and the intermediate sheet member 76, TPU is preferably used. The sheet member 75 and the intermediate sheet member 76 may have a single-layer structure or a multi-layer structure in which a plurality of resin materials are layered. In addition, the air bag 71 may be formed of, for example, a thermoplastic elastomer such as TPU and then laminated with a cloth material.

The back plate 53 is fixed to the surface of the pressing cuff 52 on the wrist 300 side with a double-sided tape, an adhesive, or the like. The back plate 53 is formed of a resin material. The back plate 53 is formed in a rectangular plate shape that is long in one direction, for example. For example, the back plate 53 may be configured to be divided, that is, may be formed by arranging a plurality of rectangular small pieces in one direction. The back plate 53 has shape followability.

Here, “shape followability” refers to a function in which the back plate 53 can be deformed in such a manner as to follow the shape of a contacted portion of the wrist 300 to be disposed. This contacted portion of the wrist 300 refers to a region of the wrist 300 that comes into contact with the back plate 53. This contact includes both direct contact with the back plate 53 and indirect contact with the back plate 53 with the sensing cuff 54 interposed therebetween.

The sensing cuff 54 is fixed to the main surface of the back plate 53 on the wrist side. The sensing cuff 54 comes into direct contact with a region of the wrist 300 where the arteries 311 and 312 are present or indirect contact with the region with the cover or the like interposed therebetween. The sensing cuff 54 is formed in a rectangular shape that is long in one direction. The sensing cuff 54 may be configured to come into direct contact with a region of the wrist 300 where one of arteries 311 and 312 are present. The sensing cuff 54 is smaller than the pressing cuff 52 in the longitudinal direction. The sensing cuff 54 is the same as or smaller than the pressing cuff 52 in the lateral direction. The sensing cuff 54 is in the same shape as that of the back plate 53 or is smaller than that of the back plate 53, in the longitudinal direction and the width direction of the back plate 53. The sensing cuff 54 compresses the region on the palm side of the hand of the wrist where the artery is present by being inflated. The sensing cuff 54 is pressed by the inflated pressing cuff 52 toward the living body side with the back plate 53 interposed therebetween.

As a specific example, the sensing cuff 54 includes one air bag 81 and a flow path body 82.

Here, the air bag 81 is a bag-like structure, and in the present embodiment, the blood pressure measurement device 1 is configured to use air with the pump 14, and thus the present embodiment will be described using the air bag. However, in a case where a fluid other than air is used, the bag-like structure may be a liquid bag and the like.

The air bag 81 is constituted in a rectangular shape that is long in one direction. For example, the air bag 81 includes a plurality of sheet members 85 and a single intermediate sheet member 86 in which an opening 86a is formed in at least a part thereof. By fixing the plurality of sheet members 85 and the single or a plurality of the intermediate sheet members 86 by thermal welding or the like, the air bag 81 is formed in a bag shape in which an inner space is partitioned by the intermediate sheet member 86 and is partitioned into two chambers that fluidly communicate with each other by the intermediate sheet member 86.

As illustrated in FIG. 6, the air bag 81 has, for example, a cuff structure in which the side walls are recessed in the 2 shape. The air bag 81 includes a pair of main walls 81a each formed by a part of a respective one of the sheet members 85 in the thickness direction, and a pair of side walls 81b each formed by an end portion in the lateral direction of a respective one of the sheet members 85 in the lateral direction. The air bag 81 may have a single cuff structure in which outer circumference edges of two sheet members 85 are welded to each other as illustrated in FIG. 8, instead of the cuff structure in which the side walls are recessed in the >shape.

The sheet member 85 forms, for example, the pair of main walls 81a in the thickness direction of the air bag 81 and a part of the pair of side walls 81b of the air bag 81. The back plate 53 is fixed to a first main wall 81a, and a second main wall 81a comes into direct contact with a region of the wrist 300 where the arteries 311 and 312 are present or indirect contact with the region with the cover or the like interposed therebetween. A central side of the side wall 81b is recessed in the 2 shape toward inward of the air bag 81 in the thickness direction of the air bag 81 (in the radial direction of the curler 51).

The plurality of sheet members 85 are welded to form the air bag 81 including the pair of main walls 81a and the pair of side walls 81b. Here, one air bag 81 may be formed by two sheet members 85, or may be formed by three or more sheet members 85, for example, four sheet members 85 constituting the main walls 81a and the side walls 81b. That is, the number of the sheet members 85 for forming the air bag 81 is not limited.

The intermediate sheet member 86 is provided at a portion located at the most inward portion of the side wall 81b. The intermediate sheet member 86 partitions the inside of the air bag 81 into two chambers (spaces) in the thickness direction of the air bag 81. Further, in the intermediate sheet member 86, a single or a plurality of the openings 86a that fluidly communicate the two chambers in the air bag 81 are formed.

For example, a thermoplastic elastomer is used as the material of the sheet member 85 and the intermediate sheet member 86. Examples of thermoplastic elastomer includes thermoplastic polyurethane-based resin, polyvinyl chloride resin, ethylene-vinyl acetate resin, thermoplastic polystyrene-based resin, thermoplastic polyolefin resin, thermoplastic polyester-based resin, and thermoplastic polyamide resin. As the thermoplastic elastomer used for the sheet member 85 and the intermediate sheet member 86, TPU is preferably used. The sheet member 85 and the intermediate sheet member 86 may have a single-layer structure or a multi-layer structure in which a plurality of resin materials are layered. In addition, the air bag 81 may be formed of, for example, a thermoplastic elastomer such as TPU and then laminated with a cloth material.

The flow path body 82 is, for example, integrally provided at a part of one edge of the air bag 81 in the longitudinal direction. The flow path body 82 is provided at the end portion of the air bag 81 near the device body 3. In addition, the flow path body 82 is formed in a shape which is long in one direction with a width smaller than the width of the air bag 81 in the lateral direction. The flow path body 82 includes a connecting portion such as a nipple on distal end thereof. The flow path body 82 is connected to the fluid circuit 24 via the connecting portion and forms a flow path between the fluid circuit 24 and the air bag 81.

The bent portion 55 is formed in at least two members among the curler 51, the pressing cuff 52, the back plate 53, and the sensing cuff 54. When the cuff structure 4 is wound around the wrist 300 and the pressing cuff 52 and the sensing cuff 54 are inflated, the bent portion 55 suppresses wrinkles from generating in the pressing cuff 52 and/or the sensing cuff 54 and controls the position where the wrinkles are generated.

In order to suppress and control the wrinkles, a single or the plurality of bent portions 55 are provided in the curler 51, the pressing cuff 52, the back plate 53, and the sensing cuff 54 which are formed. For example, the bent portion 55 formed in at least one member among at least two members of the curler 51, the pressing cuff 52, the back plate 53, and the sensing cuff 54 is formed by an opening, and the bent portion 55 formed in the other member is formed by an opening, a groove, a notch, a recessed portion, or dividing a member. Further, the shape of the bent portion 55 can be set to various shapes such as a polygonal shape including a rectangular shape, a circular shape, a linear shape, or an irregular shape. The number and arrangement of the bent portions 55 can be set as appropriate.

Hereinafter, specific examples of the bent portion 55 will be described with reference to FIGS. 5 to 23. In FIGS. 5 to 23, in describing the bent portion 55, the curler 51, the pressing cuff 52, the back plate 53, and the sensing cuff 54 may be described in the same drawing, and a plurality of reference numerals may be given to one configuration. Thus, in each drawing, the shape is illustrated by appropriately reduced, enlarged, or omitted.

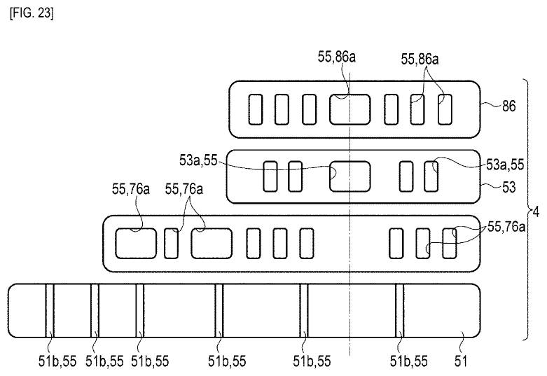

For example, as illustrated in FIGS. 5 and 22, the bent portion 55 is formed in the pressing cuff 52, the back plate 53, and the sensing cuff 54. As another example, as illustrated in FIG. 23, the bent portion 55 is formed in, for example, the curler 51, the pressing cuff 52, the back plate 53, and the sensing cuff 54.

As illustrated in FIG. 23, the bent portion 55 formed in the curler 51 is, for example, a plurality of recessed portions 51b formed on the inner surface of the curler 51. The recessed portion 51b is a groove recessed from the inner surface to the outer surface side of the curler 51. The recessed portion 51b extends, for example, in the lateral direction of the curler 51. The plurality of recessed portions 51b are disposed side by side in one direction on the inner surface of the curler 51 at equal intervals or at two or more predetermined different intervals as illustrated in FIG. 23. In addition, the shapes of the plurality of recessed portions 51b may be the same shape, or may be formed in shapes having, for example, different widths, depths, or the like. The bent portion 55 formed in the curler 51 may be an opening, a notch, or the like as long as the function of the curler 51 is not impaired. The number, shape, and arrangement of the bent portions 55 formed in the curler 51 can be set as appropriate.

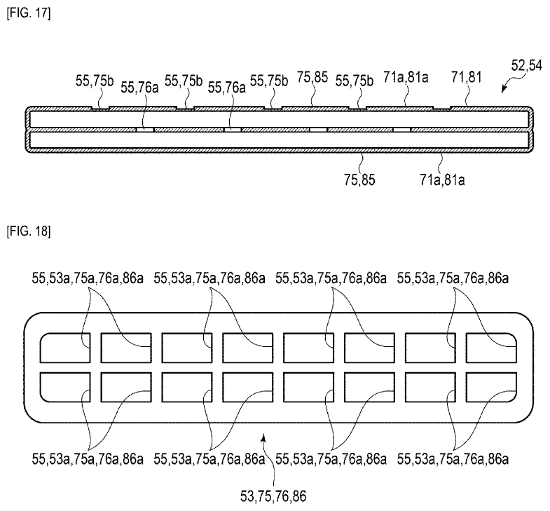

For example, as illustrated in FIGS. 5 to 7, 9 to 15, and 17 to 19, the bent portion 55 formed in the pressing cuff 52 is the single or the plurality of openings 76a formed in the intermediate sheet member 76. As illustrated in FIGS. 16 and 17, the bent portion 55 formed in the pressing cuff 52 may be a recessed portion 75b formed in the sheet member 75 in addition to the opening 76a or in place of the opening 76a which is formed in the intermediate sheet member 76. As illustrated in FIGS. 5, 7, and 19, in the case where the pressing cuff 52 is formed by layering the plurality of air bags 71, the bent portion 55 formed in the pressing cuff 52 may be the single or the plurality of openings 75a formed in the main walls 71a of the adjacent ones of the air bags 71 in addition to the opening 76a or in place of the opening 76a which is formed in the intermediate sheet member 76. As illustrated in FIGS. 9 to 15 and 18, the number, shape, and arrangement of the bent portions 55 which are the openings 76a and/or the opening 75a formed in the pressing cuff 52 can be appropriately set.

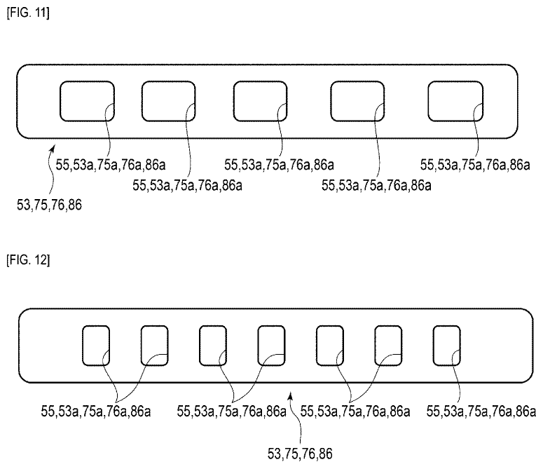

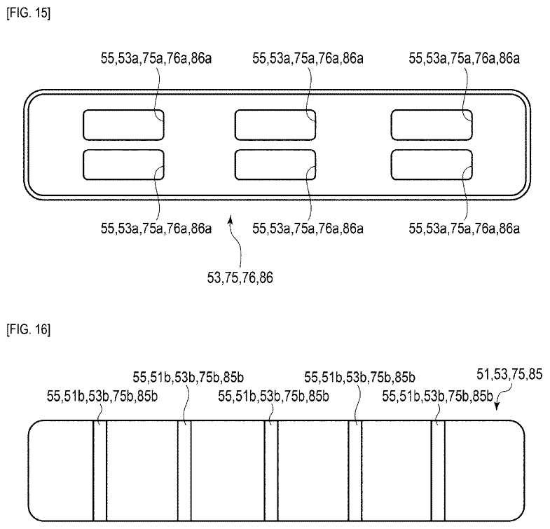

For example, the plurality of openings 75a may be formed in the same shape as illustrated in FIGS. 9 to 13 and 15, or in two or more different shapes as illustrated in FIGS. 14 and 18. The plurality of openings 75a are disposed in the longitudinal direction of the sheet member 75 at equal intervals as illustrated in FIGS. 9, 10, 12, 15 and 18, or at two or more predetermined intervals as illustrated in FIGS. 11, 13 and 14. Further, as illustrated in FIGS. 15 and 18, the plurality of openings 75a may be formed side by side in the longitudinal direction and the lateral direction of the sheet member 75.

The recessed portion 75b extends, for example, in the lateral direction of the sheet member 75. The recessed portion 75b is, for example, a welding line recessed by heating with a tool extending from the front surface of the sheet member 75 in the lateral direction of the sheet member 75. The bent portion 55 formed in the pressing cuff 52 may be a groove or a notch as long as the function of the pressing cuff 52 is not impaired. As illustrated in FIG. 16, the plurality of recessed portions 75b are disposed side by side in one direction on the inner surface of the sheet member 75 at equal intervals or at two or more predetermined different intervals. In addition, the shapes of the plurality of recessed portions 75b may be the same shape, or may be formed in shapes having, for example, different widths, depths, or the like.

The plurality of openings 76a may be formed in the same shape as illustrated in FIGS. 9 to 13 and 15, or in two or more different shapes as illustrated in FIGS. 14 and 18. The plurality of openings 76a are disposed in the longitudinal direction of the intermediate sheet member 76 at equal intervals as illustrated in FIGS. 9, 10, 12, 15 and 18, or at two or more predetermined intervals as illustrated in FIGS. 11, 13 and 14. Further, as illustrated in FIGS. 15 and 18, the plurality of openings 76a may be formed side by side in the longitudinal direction and the lateral direction of the intermediate sheet member 76.

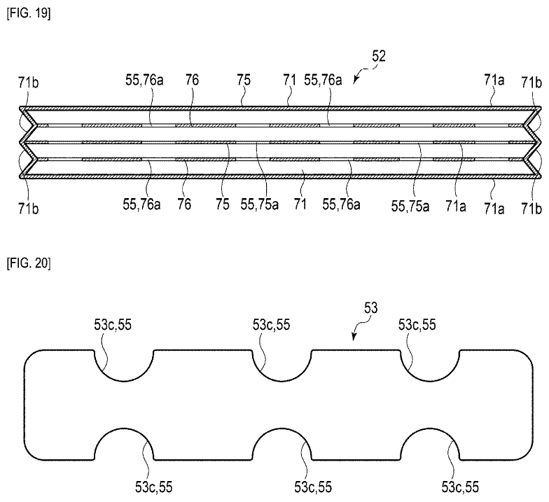

In addition, the opening 75a formed in the sheet member 75 and the opening 76a formed in the intermediate sheet member 76 may be the same as or different from each other in the number, shape, interval, and the like. For example, in the examples illustrated in FIGS. 5 and 7, the plurality of openings 75a formed in the sheet member 75 and the plurality of openings 76a formed in the intermediate sheet member 76 are disposed in different arrangement relationships, to be specific, disposed to be shifted from each other in the longitudinal direction. In addition, for example, in the example illustrated in FIG. 19, the plurality of openings 75a formed in the sheet member 75 and the plurality of openings 76a formed in the intermediate sheet member 76 are disposed in the same arrangement relationship, to be specific, disposed at the same position in the longitudinal direction, and the openings 75a and the openings 76a face each other.

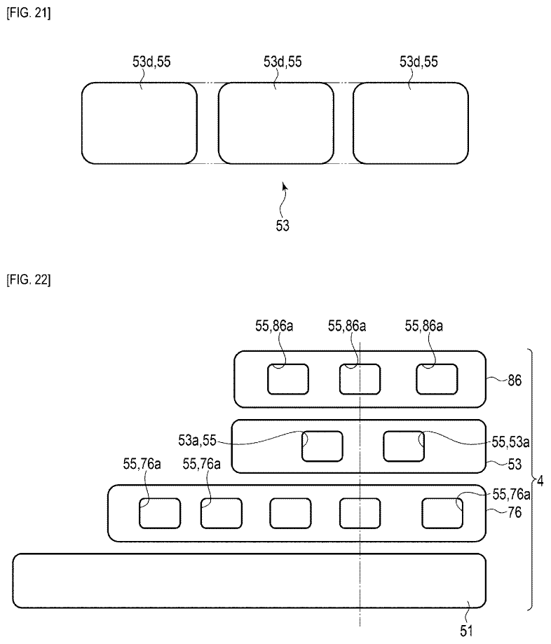

For example, as illustrated in FIGS. 5, 9 to 15, and 18, the bent portion 55 formed in the back plate 53 is a single or a plurality of openings 53a. The bent portion 55 formed in the back plate 53 may be a plurality of recessed portions 53b as illustrated in FIG. 16, or may be a plurality of notches 53c as illustrated in FIG. 20. Here, the recessed portion 53b is, for example, a groove formed in the back plate 53. As illustrated in FIG. 21, the bent portions 55 formed in the back plate 53 may be formed by dividing the back plate 53 into a plurality of small pieces 53d, arranging the divided small pieces 53d in one direction, and forming a gap between adjacent ones of the small pieces 53d. As illustrated in FIGS. 9 to 15, 18, 20, and 21, the number, shape, and arrangement of the bent portions 55 formed in the back plate 53 can be set as appropriate.

For example, the plurality of openings 53a may be formed in the same shape as illustrated in FIGS. 9 to 13, or in two or more different shapes as illustrated in FIGS. 14 and 18. The plurality of openings 53a are disposed in the longitudinal direction of the back plate 53 at equal intervals as illustrated in FIGS. 9, 10, 12, 15, 18, 20 and 21, or at two or more predetermined intervals as illustrated in FIGS. 11, 13 and 14. Further, as illustrated in FIGS. 15 and 18, the plurality of openings 53a may be formed side by side in the longitudinal direction and the lateral direction of the back plate 53.

The recessed portion 53b extends, for example, in the lateral direction of the back plate 53. The recessed portion 53b is, for example, a groove formed by a mold at the time of molding and extending from the front surface of the back plate 53 in the lateral direction of the back plate 53. As illustrated in FIG. 16, the plurality of recessed portions 53b are disposed side by side in one direction on the inner surface of the back plate 53 at equal intervals or at two or more predetermined different intervals. In addition, the shapes of the plurality of recessed portions 53b may be the same shape, or may be formed in shapes having, for example, different widths, depths, or the like.

For example, as illustrated in FIG. 20, a pair of the notches 53c is formed in the lateral direction of the back plate 53. In other words, the plurality of notches 53c are disposed symmetrically about a center line along the longitudinal direction of the back plate 53. In addition, the notches 53c are formed, for example, in an edge portion of the back plate 53 along the longitudinal direction, and are disposed side by side in one direction in the longitudinal direction of the back plate 53 at equal intervals or at two or more predetermined different intervals. As illustrated in FIG. 21, the divided small pieces 53d are disposed, for example, in one direction at equal intervals.

For example, as illustrated in FIGS. 5 to 7, 9 to 15, and 17 to 19, the bent portion 55 formed in the sensing cuff 54 is the single or the plurality of openings 86a formed in the intermediate sheet member 86. The bent portion 55 formed in the sensing cuff 54 may be a recessed portion 85b formed in the sheet member 85 in addition to the opening 86a or in place of the opening 86a which is formed in the intermediate sheet member 86.

For example, the plurality of openings 86a may be formed in the same shape as illustrated in FIGS. 9 to 13 and 15, or in two or more different shapes as illustrated in FIGS. 14 and 18. The plurality of openings 86a are disposed in the longitudinal direction of the intermediate sheet member 86 at equal intervals as illustrated in FIGS. 9, 10, 12, 15 and 18, or at two or more predetermined intervals as illustrated in FIGS. 11, 13 and 14. Further, as illustrated in FIGS. 15 and 18, the plurality of openings 86a may be formed side by side in the longitudinal direction and the lateral direction of the intermediate sheet member 86.

The recessed portion 85b extends, for example, in the lateral direction of the sheet member 85. The recessed portion 85b is, for example, a welding line recessed by heating with a tool extending from the front surface of the sheet member 85 in the lateral direction of the sheet member 85. The bent portion 55 formed in the sensing cuff 54 may be a groove or a notch as long as the function of the sensing cuff 54 is not impaired. As illustrated in FIG. 16, the plurality of recessed portions 85b are disposed side by side in one direction on the inner surface of the sheet member 75 at equal intervals or at two or more predetermined different intervals. In addition, the shapes of the plurality of recessed portions 85b may be the same shape, or may be formed in shapes having, for example, different widths, depths, or the like.

For example, as illustrated in FIG. 22, in the cuff structure 4 configured as described above, the bent portions 55 formed in the members may be disposed to be shifted from each other in the longitudinal direction of the curler 51, the pressing cuff 52, the back plate 53, and the sensing cuff 54 such that the bent portions 55 partially overlap each other in the lamination direction of the curler 51, the pressing cuff 52, the back plate 53, and the sensing cuff 54. In addition, in the cuff structure 4, the bent portions 55 formed in the members may be disposed to be shifted from each other in the longitudinal direction of the curler 51, the pressing cuff 52, the back plate 53, and the sensing cuff 54 such that the bent portions 55 do not overlap each other in the lamination direction of the curler 51, the pressing cuff 52, the back plate 53, and the sensing cuff 54.

In addition, for example, as illustrated in FIG. 23, in the cuff structure 4, some of the bent portions 55 formed in the members may be disposed at the same position in the longitudinal direction of the curler 51, the pressing cuff 52, the back plate 53, and the sensing cuff 54 such that the some of the bent portions 55 completely overlap each other in the lamination direction of the curler 51, the pressing cuff 52, the back plate 53, and the sensing cuff 54 and the rest of the bent portions 55 formed in the members may be partially or completely shifted from each other in the longitudinal direction.

In addition, in the cuff structure 4, the bent portions 55 formed in the members may be disposed at the same position in the longitudinal direction of the curler 51, the pressing cuff 52, the back plate 53, and the sensing cuff 54 such that all of the bent portions 55 overlap each other in the lamination direction of the curler 51, the pressing cuff 52, the back plate 53, and the sensing cuff 54.

The band 5 brings the cuff structure 4 into close contact with the wrist 300 and fixes the cuff structure 4. The band 5 is provided on one of the first curved portion 62 and the second curved portion 63 of the curler 51. The band 5 is formed, for example, in a band-like shape. As illustrated in FIGS. 2 and 3, the band 5 is inserted into the loop portion 31a provided on the outer case 31 and is folded back. The band 5 includes, for example, a pair of hook-and-loop fasteners 5a in which a hook is formed on a first side and a loop is formed on a second side, and when the pair of hook-and-loop fasteners 5a engage with each other, the band 5 whose end portion side is inserted into the loop portion 31a is fixed.

In the present embodiment, the band 5 is provided at a distal end of the second curved portion 63, and the loop portion 31a is provided on the outer surface of the outer case 31 on the side where the first curved portion 62 is provided.

The band 5 may be configured to include a first band which is a so-called parent band and includes a buckle, and a second band which is a so-called sword tip and includes a plurality of small holes into which a prong of the buckle is inserted.

Next, an example of the power transmission device 100 that transmits power to the charging circuit unit 21 of the device body 3 will be described. As illustrated in FIG. 4, the power transmission device 100 includes a power source 101, a power transmission unit 102, and an antenna unit 103. The power source 101 is, for example, an AC adapter or the like connected to a commercial power source or the like. The power source 101 converts AC power input from the commercial power source into DC power and supplies the DC power to the power transmission unit 102.

The power transmission unit 102 generates AC power as transmission power from the DC power supplied from the power source 101, and supplies the AC power to the antenna unit 103. For example, the power transmission unit 102 generates AC power having a frequency that is the same or substantially the same as a resonance frequency of a power transmission resonance circuit of the antenna unit 103.

The antenna unit 103 is, for example, a transmitter coil as the power transmission resonance circuit. A power transmission surface of the antenna unit 103 is formed in a planar shape. The antenna unit 103 transmits power to the antenna unit 211 of the device body 3. The antenna unit 103 includes, for example, a resonance capacitor and constitutes the power transmission resonance circuit.

According to the blood pressure measurement device 1 configured as described above, the bent portion 55 is formed in at least two of the curler 51, the air bag 71 of the pressing cuff 52, the back plate 53, and the air bag 81 of the sensing cuff 54, which are the plurality of members constituting the cuff structure 4. In addition, among at least two members of the curler 51, air bag 71 of pressing cuff 52, back plate 53, and air bag 81 of sensing cuff 54 in which bent portion 55 is formed, the bent portion 55 formed in at least one member includes the opening, and the bent portions 55 formed in the rest of the members include the opening, the division, the notch, or the groove.

With these configurations, in the bent portion 55, the intensity, here the bending intensity, of the portions where the bent portions 55 of the curler 51, the air bag 71 of the pressing cuff 52, the back plate 53, and the sensing cuff 54 are formed is lower than the intensity of the portion where the bent portion 55 is not formed. Thus, the portion where the bent portion 55 is provided is more easily bent and deformed than the portion where the bent portion 55 is not provided, and becomes a base point at which the wrinkles are generated in the air bag 71 of the pressing cuff 52 and the air bag 81 of the sensing cuff 54. Here, the wrinkles generated in the air bags 71 and 81 are generated by factors such as a difference between the inner and outer circumferences that is generated when the air bag 71 is inflated in a curved state, and the shape of the wrist 300. However, the generated wrinkles can be controlled by the bent portion 55. For example, the position at which the wrinkles are generated can be adjusted by the number, arrangement, and shape of the bent portions 55, and thus the number of the wrinkles generated in the air bag 71 of the pressing cuff 52 and the air bag 81 of the sensing cuff 54 can be adjusted, and the depth of each wrinkle can be adjusted by the number of the wrinkles.

In this way, the cuff structure 4 can generate the wrinkles in the air bags 71 and 81 at a desired number, position, and depth by adjusting the bending intensity by the bent portion 55. Further, as illustrated in FIG. 22, by shifting the bent portions 55 of the respective members in the longitudinal direction of the respective members so that the bent portions 55 are alternately disposed, the wrinkles can be finely dispersed. Further, as illustrated in FIG. 23, by overlapping the bent portions 55 of the respective members, the wrinkle can also be generated at a predetermined target position. That is, even when the characteristics such as bending intensity of the cuff structure 4 are different, desired wrinkles can be generated by adjusting the number, arrangement and shape of the bent portions 55.

Thus, even when the air bags 71 and 81 have a relatively thick cuff structure in which the side wall is recessed in the 2 shape, the wrinkles can be controlled. As described above, the cuff structure 4 can prevent the spaces in the air bags 71 and 81 from being divided by the wrinkles due to the wrinkles becoming excessively deep or the like, and thus the measurement accuracy of the blood pressure measurement device 1 can be stabilized. That is, the blood pressure measurement device 1 can suppress variations in the generation of the wrinkles due to a state of the cuff structure 4 at the time of attachment to the wrist 300 and the shape of the wrist 300, and can suppress variations in the measurement accuracy of the blood pressure caused by the wrinkles.

In the blood pressure measurement device 1, it is preferable to disperse the wrinkles by setting the number, arrangement, and shape of the bent portions 55 so that the cuff structure 4 is intentionally bent to a large degree at a position where the curvature is relatively large, such as the boundary (ridge) between the fixed portion 61 and the first curved portion 62 and the boundary (ridge) between the fixed portion 61 and the second curved portion 63, or so that the wrinkles are less likely to be generated in the vicinity of the arteries 311 and 312 that are susceptible to a decrease in compression efficiency. As a result, the cuff structure 4 can reduce the influence on the stress concentration and the decrease in compression efficiency.

In addition, the member and the position at which the bent portion 55 is provided, and the number, shape, and arrangement of the bent portions 55 can be any selected combination. Thus, in the cuff structure 4, control of the wrinkles can be achieved by setting the bent portion 55 in terms of ease of manufacture, cost, and the like.

In addition, when the air bags 71 and 81 have a cuff structure in which the side walls are recessed in the 2 shape, the air bags 71 and 81 can be prevented from being laterally bulged, and thus high compression efficiency can also be obtained. In addition, when the openings 76a and 86a are provided in the intermediate sheet members 76 and 86 and the lateral bulge is generated in the air bags 71 and 81, the lateral bulge can be suppressed by setting the shapes of the openings 76a and 86a to be small, and when the lateral bulge is not generated, the openings 76a and 86a can be set to be large. In addition, the sizes of the openings 76a and 86a can be partially changed according to the size, depth, and the like of the wrinkles to be generated. As described above, in the cuff structure 4, the intensities of the intermediate sheet members 76 and 86 are adjusted by the shape of the openings 76a and 86a, and the like, and the bent portion 55 formed in each member can be set with a high degree of flexibility based on the relationship between suppression of generation of the lateral bulge and control of the wrinkles when the air bags 71 and 81 have the cuff structure in which the side wall is recessed in the >shape.

Next, an example of an evaluation result of the blood pressure measurement device 1 according to the embodiment will be described by using FIG. 24. As the evaluation test, the blood pressure measurement device 1 according to the embodiment and the blood pressure measurement devices (cuff structures) of Comparative Example 1 and Comparative Example 2 were inflated, and wrinkles generated in the sensing cuff 54 were evaluated.

In the blood pressure measurement device 1 according to the embodiment used in the evaluation test, the sheet member 75 of the sensing cuff 54 was provided with the plurality of recessed portions 75b which were welding lines as the bent portions 55 at equal intervals of 8 mm, and the intermediate sheet member 76 was provided with the plurality of openings 76a as the bent portions 55. The sensing cuff 54 was formed of TPU and the front surface thereof was laminated with a cloth material.

In the blood pressure measurement device of Comparative Example 1, the sheet member 75 of the sensing cuff 54 was provided with the plurality of recessed portions 75b which were welding lines as the bent portions 55 at equal intervals of 8 mm, and the intermediate sheet member 76 was not provided with the plurality of openings 76a as the bent portions 55. In the blood pressure measurement device of Comparative Example 1, the surface of the sensing cuff 54 was formed of TPU without providing a cloth material.

In the blood pressure measurement device of Comparative Example 2, the sheet member 75 of the sensing cuff 54 was provided with the plurality of recessed portions 75b which were welding lines as the bent portions 55 at equal intervals of 8 mm, and the intermediate sheet member 76 was not provided with the plurality of openings 76a as the bent portions 55. In the blood pressure measurement device of Comparative Example 2, the sensing cuff 54 was formed of TPU, and the front surface thereof was laminated with the cloth material.

As illustrated in FIG. 24, when the cuff structure 4 of the blood pressure measurement device 1 according to the embodiment was inflated, the wrinkles generated in the sensing cuff 54 could be uniformly dispersed. As a result, in the cuff structure 4 according to the embodiment, for example, the opening 76a of the intermediate sheet member 76 as at least one member is provided as the bent portion 55, and the sheet member 75 is combined as another member, so that the wrinkles could be uniformly and dispersedly generated.

On the other hand, in the cuff structure of the blood pressure measurement device of Comparative Example 1, the sensing cuff 54 did not have the cloth laminate, and thus the sensing cuff 54 was softer than the sensing cuffs 54 of the cuff structures of the embodiment and Comparative Example 2, and the intermediate sheet member did not include an opening, and thus the wrinkles varied in size and could not be uniformly dispersed.

In addition, the cuff structure of the blood pressure measurement device of Comparative Example 2 included the cloth laminate, so that the sensing cuff 54 was harder than that of Comparative Example 1, and thus the wrinkles were not generated in some of the recessed portions 75b (welding lines) among the plurality of recessed portions 75b.

From the result of such an evaluation test, it is clear that the blood pressure measurement device 1 according to the present embodiment can control the generated wrinkles.

As described above, according to the blood pressure measurement device 1 according to the present embodiment, the bent portion 55 including the opening is formed in at least two members constituting the cuff structure 4, and thus the generated wrinkles can be controlled.

Note that the present invention is not limited to the embodiments described above. That is, the cuff structure 4 may have a configuration in which the bent portion 55 is provided in at least two members, and thus, the cuff structure 4 may have a configuration including one or more air bags and one or more other members that are included in the air bags or are laminated on the air bags. Thus, for example, the cuff structure 4 may be configured to include only one cuff, or may be configured to further include another cuff in addition to the pressing cuff 52 and the sensing cuff 54.

That is, the present invention is not limited to the above-described embodiment, and various modifications can be made in an implementation stage without departing from the gist thereof. Furthermore, each of the embodiments may be carried out as appropriate in a combination as much as possible, and combined effects can be obtained in such a case. Furthermore, the inventions at various stages are included in the above-described embodiment, and the various inventions can be extracted in accordance with appropriate combinations in the plurality of disclosed constituent elements. Note that the present invention is not limited to the above-described embodiments, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 Cuff structure
5 Band
5a Hook-and-loop fastener
11 Housing
12 Display unit
13 Operation unit
14 Pump
15 Acceleration sensor
16 Valve
17 Pressure sensor
18 Battery
19 Communication unit
20 Biometric sensor
21 Charging circuit unit
22 Memory
23 Processor
24 Fluid circuit
31 Outer case
31a Loop portion
32 Windshield
33 Back lid
41 Button
43 Touch panel
51 Curler
51a Decorative sheet
51b Recessed portion
52 Pressing cuff
53 Back plate
53a Opening
53b Recessed portion
53c Notch
53d Small piece
54 Sensing cuff
55 Bent portion
61 Fixed portion
62 First curved portion
63 Second curved portion
71 Air bag
71a Main wall
71b Side wall
75 Sheet member
75a Opening
75b Recessed portion
76 Intermediate sheet member
76a Opening
81 Air bag
81a Main wall
81b Side wall
82 Flow path body
85 Sheet member
85b Recessed portion
86 Intermediate sheet member
86a Opening
100 Power transmission device
101 power source
102 Power transmission unit
103 Antenna unit
211 Antenna unit
212 Power receiving unit
213 Charging unit
300 Wrist
311 Radial artery
312 Ulnar artery

1. A cuff structure for use in a blood pressure measurement device, the cuff structure comprising:
one or more air bags that are inflated by a fluid;
one or more members that are included in the air bags or are laminated on the air bags;
a curler that is the member and is curved following a shape of a site of a living body, to be attached thereto in a circumferential direction;
a pressing cuff that is fixed to an inner circumferential surface of the curler and includes the air bag;
a back plate that is the member and is fixed to an inner circumferential surface of the pressing cuff; and
a sensing cuff that is fixed to an inner circumferential surface of the back plate and includes the air bag, wherein
the air bag and at least two of the one or more members are provided with bent portions, one of which is formed by an opening or a division and the other of which is formed by an opening, a division, a notch, a recessed portion, or a groove,
the air bag includes an intermediate sheet member and has a cuff structure in which a side wall is recessed, and
the bent portion formed in the air bag includes one or more openings formed at least in the intermediate sheet member.

2. The cuff structure according to claim 1, wherein
the bent portion formed in the back plate is an opening, a division, a notch, or a groove formed in the back plate.

3. The cuff structure according to claim 1, wherein
the curler, the pressing cuff, the back plate, and the sensing cuff are formed to be long in one direction, and
a plurality of the bent portions provided in at least two of the curler, the pressing cuff, the back plate, and the sensing cuff are provided side by side in the one direction.

4. The cuff structure according to claim 3, wherein
the bent portions provided in at least two of the curler, the pressing cuff, the back plate, and the sensing cuff are disposed to at least partially overlap the bent portions adjacent to each other in a lamination direction and/or disposed to be shifted from the bent portions adjacent to each other in the lamination direction in the one direction.

5. A blood pressure measurement device comprising:
the cuff structure according to claim 1;
a device body fixed to the cuff structure; and
a band provided in the device body and configured to fix the cuff structure.
